SEQUENCE LISTING (15 sequences)

SEQ ID NO: 1 - DNA, 6 bp, Artificial Sequence
  DNA recognition sequence
gtmkac

SEQ ID NO: 2 - DNA, 4 bp, Artificial Sequence
  DNA recognition sequence
agct

SEQ ID NO: 3 - DNA, 6 bp, Artificial Sequence
  DNA recognition sequence
raatty

SEQ ID NO: 4 - DNA, 6 bp, Artificial Sequence
  DNA recognition sequence
cycgrg

SEQ ID NO: 5 - DNA, 5 bp, Artificial Sequence
  DNA recognition sequence
ggwcc

SEQ ID NO: 6 - DNA, 6 bp, Artificial Sequence
  DNA recognition sequence
yacgtr

SEQ ID NO: 7 - DNA, 6 bp, Artificial Sequence
  DNA recognition sequence
cgrycg

SEQ ID NO: 8 - DNA, 6 bp, Artificial Sequence
  DNA recognition sequence
rgatcy

SEQ ID NO: 9 - DNA, 6 bp, Artificial Sequence
  DNA recognition sequence
rgcgcy

SEQ ID NO: 10 - DNA, 5 bp, Artificial Sequence
  DNA recognition sequence
cgwcg

SEQ ID NO: 11 - DNA, 4 bp, Artificial Sequence
  DNA recognition sequence
ttaa

SEQ ID NO: 12 - DNA, 6 bp, Artificial Sequence
  DNA recognition sequence
rcatgy

SEQ ID NO: 13 - DNA, 4 bp, Artificial Sequence
  DNA recognition sequence
gtac

SEQ ID NO: 14 - DNA, 6 bp, Artificial Sequence
  DNA recognition sequence
ctryag

SEQ ID NO: 15 - DNA, 6 bp, Artificial Sequence
  DNA recognition sequence
cgwwgg

(19) United States
(12) Patent Application Publication
(10) Pub. No.: US 2013/0004951 A1
(43) Pub. Date: Jan. 3, 2013

(54) BULKED MUTANT ANALYSIS

(75) Inventors: Jeroen Stuurman, Wageningen (NL); Feyruz Yalçin, Wageningen (NL)

(73) Assignee: Keygene N.V.

(21) Appl. No.: 13/516,591

(22) PCT Filed: Dec. 17, 2010
(86) PCT No.: PCT/NL10/50860

§ 371 Date: Sep. 7, 2012

Related U.S. Application Data
(60) Provisional application No. 61/287,927, filed on Dec. 18, 2009.

(30) Foreign Application Priority Data
Dec. 24, 2009 (NL) 2004019

(51) Int. Cl. C12Q 1/68

(52) U.S. Cl. 435/6.11

(57) ABSTRACT

The current invention relates to a new strategy for identification, and optional isolation, of a nucleic acid sequence that is expressed in an organism and that is causally related to a particular phenotype (trait of a character) of said organism. With the method of the current invention it has become possible to, in contrast to known methods in the art, efficiently enrich, identify, isolate and/or clone genes in, for example, organism like (crop) plants for which no or only limited information with respect to the genome is available.

### BACKGROUND ART

Cloning genes responsible for organismal traits that are defined only by phenotype (so-called forward gene cloning) has been a longstanding challenge in genetics and biotechnology. In particular in the field of (crop) plants, rapid and economical methods for forward gene cloning are in high demand. The current quest is to find methods that enhance the speed and reduce the costs of forward gene isolation, and in particular to extend the range of species in which gene cloning can be practiced. Gene cloning is especially cumbersome in species with large or uncharacterized genomes that contain large amounts of repetitive DNA. Such species are lettuce, pepper, onion, leek, bean, wheat, rye, barley, and many others.

The objective in forward gene cloning is the identification of those genes that are known only from a phenotype and for which no or only very limited molecular information or no sequence information is available. The starting point in forward genetics can, for example, be naturally occurring phenotypic variants or artificially induced mutants.

Essentially, two groups of methods are being recognized for forward gene cloning: mapping strategies and tagging strategies. These strategies are complementary, each with its inherent limitations.

In mapping strategies, phenotypes are pinpointed to the smallest-most segment of a chromosome by marker-assisted meiotic recombination mapping. Map-based cloning is a laborious procedure and is in particular employed in a small group of model species for which extensive genomic information, preferably a whole genome DNA sequence, is available.

Theoretically, map-based cloning is a universally applicable procedure for any organism that reproduces through a sexual cycle (Peters et. al. (2003) Trends in Plant Science Vol. 8 No. 10 pp 484-491). In practice, however, there are fundamental biological constraints. Most importantly, map-based cloning depends critically on a good frequency of meiotic recombination in the area of the gene of interest. Secondly, it becomes increasingly difficult in poorly characterized large genomes, for example in plants like lettuce, pepper, onion, and many others, where repetitive DNA makes up a large portion of the genome, hindering the unequivocal mapping of DNA markers and the assembly of large contiguous DNA sequences that span a genetic interval.

In classical tagging strategies, well-characterized biological insertion mutagens (transposons or T-DNA insertions) have been used as a vehicle to clone genes. Insertion of a transposable element into a gene can lead to loss- or gain-of-function, changes in expression pattern, or can have no effect on gene function, depending on whether the insertion took, for example, place in coding or non-coding regions of the gene. Genes responsible for any newly arising phenotype by definition flank the insertion element, and are thus cloned by retrieving the insertion sequences along with flanking pieces of genomic DNA.

Tagging with the help of insertion mutagens is theoretically a preferable approach to clone genes because it is fast, independent of meiotic recombination, and requires no previous genomic resources such as genetic maps or extensive sequence information (See Maes et al. Trends Plant Sci. 1999 March; 4(3):90-96.). Cloning mutated genes by tagging is not hindered by repetitive genomic sequences or by genome size.

The tagging approach has been successful in a handful of model organisms, in which natural gene tagging (transposon) systems were known, or where large numbers of individuals could be transformed with randomly integrating DNA (T-DNA) (see for example Settles et. al, BMC Genomics 2007, 8:116, using maize). Outside of this small group of species, tagging cannot or hardly be used either due to the absence of known insertion elements, or due to logistic constraints on population size. Logistically, tagging requires populations of many thousands of individuals in order to find one or a few specific mutants, as the number of insertion sequences per genome is low (1-200).

It is one of the goals of the present invention to provide for a new method, and the use thereof, which allows for the efficient identification of nucleic acids that are involved in the manifestation of a particular phenotype. The method is employable in any (plant) species that can be artificially crossed and manipulated, without the need of extensive meiotic recombination mapping and pre-existing genomic DNA sequence knowledge of the species. Other goals of the present invention will become apparent from the description of the invention, the embodiments and the claims.

### DEFINITIONS

In the following description and examples several terms are used. In order to provide a clear and consistent understanding of the specification and claims, including the scope to be given to such terms, the following definitions are provided. Unless otherwise defined herein, all technical and scientific terms used have the same meaning as commonly understood by one of ordinary skill in the art to which this invention belongs. The disclosures of all publications, patent applications, patents and other references are incorporated herein in their entirety by reference.

Allele: One of at least two alternative forms of a gene that can have the same place on homologous chromosomes and are responsible for alternative traits. A non-limitative example is a gene for blossom colour in a flower—a single gene might control the colour of the petals, but there may be several different versions or alleles of the gene. One version might result in red petals, while another might result in white petals. The resulting colour of an individual flower will depend on which two alleles it possesses for the gene and how the two interact.

Character: Relates to a phenotypical quality of an organism. A character can manifest itself in different traits. For example, the organism can be a plant, having flower colour as a character, and the red or white flowers being the traits A and B of the character. Within the current invention, the character (or trait) can be any, as long as members of the organism having a first trait of the character can be phenotypically distinguished from members of the organism having a second trait of the character. This is not limited to only differences that can be directly observed by inspection of an organism, but also includes characters/traits that can become apparent upon further analysis of the organism, for example upon analysis of the resistance to certain circumstances (e.g to certain diseases), or upon analysis of the presence of particular metabolites in such organism.

“Expression of a gene” or “expressed nucleic acid” within the context of the current invention refers to the process wherein a DNA region, which is operably linked to appropriate regulatory regions, particularly a promoter, is transcribed into an RNA, which is biologically active, i.e. which is capable of being translated into a biologically active protein or peptide or which is active itself.

A “Gene” is a DNA sequence comprising a region (transcribed region), which is transcribed into an RNA molecule (e.g. an mRNA) in a cell, operably linked to suitable transcription regulatory regions (e.g. a promoter). A gene may thus comprise several operably linked sequences, such as a promoter, a 5′ non-translated leader sequence (also referred to as 5′UTR, which corresponds to the transcribed mRNA sequence upstream of the translation start codon) comprising e.g. sequences involved in translation initiation, a (protein) coding region (cDNA or genomic DNA) and a 3′ non-translated sequence (also referred to as 3′ untranslated region, or 3′UTR) comprising e.g. transcription termination sites and polyadenylation site.

Isogenic meansgenetically identical. Individual cells within an isogenic population are typically the progeny of a single ancestor, having equal genetic make-up. Within the current invention, “isogenic” is to be construed that at the level of cDNA the individual members are 95-99%, even 100% identical except for any point mutation that might arise as a consequence of natural variation, or, in a preferred embodiment of the invention, from a mutagenic treatment. Indeed the term isogenic is well-known and understood by the skilled person.

Mutagenesis or mutagenic treatment: The terms relate herein to a treatment leading to the introduction of changes in nucleic acid, genes, or genomes, for example leads to the introduction of point mutations and/or insertion or deletion of up to 10 consecutive nucleotides.

Nucleic acid: a nucleic acid according to the present invention may include any polymer or oligomer of pyrimidine and purine bases, preferably cytosine, thymine, and uracil, and adenine and guanine, respectively (See Albert L. Lehninger, Principles of Biochemistry, at 793-800 (Worth Pub. 1982) which is herein incorporated by reference in its entirety for all purposes). The nucleic acids may be DNA, including cDNA, or RNA, or a mixture thereof, and may exist permanently or transitionally in single-stranded or double-stranded form, including homoduplex, heteroduplex, and hybrid states.

Sequencing: The term sequencing refers to determining the order of nucleotides (base sequences) in a nucleic acid sample, e.g. DNA or RNA.

Trait: In biology, a trait relates to any phenotypical distinctive character of an individual member of an organism in comparison to (any) other individual member of the same organism. Within the context of the current invention the trait can be inherited, i.e. be passed along to next generations of the organism by means of the genetic information in the organism. Non-limitative examples of traits include, for example, traits that can be observed visually or those that can be observed by analysis (for example for the presence or level of a metabolite).

“Trait of the same character” or “trait of said character”: anyone of a group of at least two traits that exist (or became apparent) for a character. For example, in case of the character “colour of the flower”, phenotypical manifestations might comprise blue, red, white, and so on. In the above example blue, red and white are all different traits of the same character.

### DETAILED DESCRIPTION OF THE INVENTION

The above mentioned goals are surprisingly solved by providing for a method as described in the accompanying claims.

More specifically, there is provided a method for identification, and optional isolation, of an expressed nucleic acid sequence that is associated with a character of an organism, characterized in that the method comprises the following steps
a) Providing at least two members of said organism having a trait A of said character, and wherein said members having trait A are derived, preferably independently derived, from isogenic member(s) of said organism having trait B, and wherein trait A and trait B are different;
b) Obtaining cDNA from each of the members of step a) having trait A and determining nucleotide sequences of at least part of, preferably of each of the individual cDNA's obtained from the members having trait A;
c) Determining single nucleotide polymorphism positions in each individual cDNA of members having trait A by comparison to corresponding cDNA of other members having trait A;
d) Identifying individual cDNAs in which at least 2 of the members having trait A, preferably all of the members having trait A, contain at least one single nucleotide polymorphism, and preferably where these single nucleotide polymorphisms are on a different position in the cDNA for said at least two, preferably all members having trait A;
e) Optionally, verifying for each of the cDNAs indentified in step d) that it is associated with a character of an organism.

Optionally and preferably, the method can comprise the step (f) of identifying individual cDNAs from each of the cDNAs identified in d) in which at least two, preferably all members having trait A have at least one non-synonymous single nucleotide polymorphism.

Next, in a preferred embodiment the method may comprise the step (g) of identifying individual cDNAs from each of the cDNAs identified in the previous described step (f) in which at least two, preferably all members having trait A have exactly one non-synonymous single nucleotide polymorphism

Also, the method may comprise the step (h) of listing of all individual cDNAs identified in performed steps (d), (f) and/or (g), and ranking them according to the number of performed steps (d), (f) and/or (g) that they comply with (most compliance is highest rank).

The method may also comprise, preferably, identifying the expressed nucleic acid sequences from which the cDNAs identified are derived and, optionally, cloning the gene comprising the expressed nucleic acid sequences.

The current invention is based on the realization that the above mentioned problems with current forward cloning strategies can be solved by the method according to the invention, and that overcomes the limitations of methods based on tagging with biological mutagens, by using non-biological mutagens that can be applied to any organism, and which produces thousands of well-detectable mutations per genome to lift logistical population constraints as much as possible, in combination with sequencing of the transcriptomes (cDNA) obtained from a mutant collection (of at least 2 members) showing a phenotype (trait A), and based thereupon identifying any gene which carries at least one single nucleotide polymorphism, preferably at least one non-synonymous single nucleotide polymorphism, in each of the members of said mutant collection (but not necessarily on the same position within said gene/cDNA). In addition, the mutant collection can be obtained from natural variation.

In addition to mutation induced by the treatment with, for example, a mutagen, also spontaneous mutants that occur in a further isogenic population can be used. In other words, in case in an isogenic population having a trait B at least two members with a trait A are found (e.g. due to spontaneous mutation), the gene responsible for the character and trait can be identified with the method as disclosed herein.

It was realized that genes in any organism can be tagged (and thereby may manifest a distinct phenotype) with chemically induced mutations like point mutations. However, a major problem lies in how to detect such small and random mutations (tags) in the whole genome, as such mutation does not qualify for any form of specific PCR amplification. Moreover, chemically induced mutations are randomly introduced in the genome with high frequency. Consequently, many genes might concurrently comprise mutations in comparison to the genes of the original species, further complicating the correct identification of the gene responsible for an observed phenotype, or for showing a particular trait.

The inventors have realized that the solution can come from creating a collection of independent and distinct mutant alleles at the locus to be cloned (but which is yet unknown), and then re-sequencing all cDNAs from this collection multiple times. When comparing (all) cDNA sequences in this collection, one cDNA in the mutant pool will show consistent mutation. This is extremely likely a gene underlying the mutant phenotype.

In other words, and for example, one can treat (seeds of) an isogenic population manifesting a particular trait B, for example red flowers, with a mutagen inducing point mutations in the genome. As a consequence of the treatment with said mutagen random mutations are introduced in the genome of each treated members. Normally, after the treatment most of the treated members will still manifest the particular trait B. In those members the mutations introduced by the treatment with the mutagen did not alter the trait. In other words, in these members no genes related to the character of the trait are mutated such that the trait changes (for example into a trait A, being, for example, yellow flowers). However, in some members the mutagen may have induced mutations in a gene underlying the observed traits in such manner that the originally trait B is not manifested anymore, but trait A is manifested in those members.

Due to the random character of the mutagen, and in case at least two members, preferably more members, having trait A are observed, it is highly likely that the mutations induced by the mutagen and responsible for the change from trait B to trait A is, although positioned in the same gene, not at the same position in said gene. For example, in a first member having trait A, the mutation may be positioned at nucleotide R, in a second member having trait A, the mutation may be positioned at nucleotide S, and in a third, fourth and fifth member having trait A, the mutation may be positioned at, respectively, positions T, U, and V (with R, S, T, U, and V being different positions with the gene). In other words, these members all show, when compared to each other, a mutation at different positions in the responsible gene. At the same time, although the members showing trait A also carry mutations at other locations throughout the genome, the chance that another expressed gene is modified in each of the members now showing trait A is virtually zero.

By now comparing the cDNA's obtained from the members having trait A with each other, a cDNA may be identified that in each of the members having trait A carry a modification of at least one nucleotide, and which is positioned at a different position within said cDNA in at least two of said members. Said cDNA is responsible for the observed traits of the same character.

In the method according to the invention, pools are made from an (induced) allelic series in an otherwise isogenic genetic background. The method according to the invention detects SNPs that are located within the gene to be cloned.

The method is applicable to all species that can be artificially hybridized and mutagenised, including such notoriously cumbersome crops as peppers and onions, is independent of the presence of a genetic map, and will work in genomes of any size and complexity.

With the method according to the invention it has thus now become possible to identify among a large number of genes those genes that are likely candidates to be involved in or responsible for an observed trait. In practice, when applying the method according to the invention, the group of likely candidates can rapidly and reliably be limited to, for example 10, 5, 3, 2, or even 1 candidate-gene. With the method according to the invention, rapid identification of genes responsible or involved in the manifestation of a particular trait has now become possible.

In more detail, in the method according to the invention at least two individual members of an organism having a particular phenotype (or having a particular trait A) are compared. The at least two members are derived from isogenic organism(s) having (a different) trait B.

For the invention it is important that the members of the organism having trait A or trait B are (in the case of trait A) derived from (e.g. by mutagen treatment or by spontaneous mutations) or are (in the case of trait B) isogenic members of said organism, in other words the organisms have the same genetic background (for example are derived from the same inbred strain). The individual members having trait A or trait B are isogenic, i.e. have the same genetic background, except for changes that were introduced into the genetic material due to natural variation or, in a preferred embodiment of the invention, due to a mutagenic treatment.

It is in these differences in the genetic material between the members having trait A and the members having trait B that the observed phenotype is comprised. As a consequence of the observed change in the character (from trait A to trait B) the members having trait A must have been modified such that they now express at least one nucleic acid that is different from the nucleic acids expressed by the members of the organism having trait B, and which nucleic acid is associated with the character of the organism, of which trait A and trait B are phenotypical forms.

Preferably, the at least two members having trait A are independently derived from isogenic members of said organism having trait B.

The term “independently derived” indicates that the at least two members having a trait A are members that independently from each other obtained said trait A, for example as a consequence of mutagen treatment. In case the organism is a plant, the at least two members might each have acquired the trait A upon mutagen-treatment of for example the seed from which the plant grows. The trait is in such example introduced in a first member independently from any further member manifesting trait A. In other words, preferably the members having trait A do not share a common predecessor during, for example, the last 3, 4, 5, 6, 7, 8, 9 or 10 generations, already manifesting said trait A. Preferably all members are independently derived from isogenic members of said organism having trait B.

As mentioned above, as manifested by the presence of the two different traits, the members having trait A express at least one nucleic acid that is different from the nucleic acids expressed by other members having trait B, and which nucleic acid is associated with the character of the organism, of which trait A and trait B are phenotypical forms.

In order to detect such tagged (by, for example, a point mutation) nucleic acid, in the method of the current invention, cDNA, preferably total cDNA, from each of the members of step a) having trait A is obtained and the nucleotide sequence of the individual cDNA's, preferably each of the cDNA's obtained from said members having trait A is determined. In other words, the transcriptomes, preferably total transcriptomes, of all members having trait A are compared with the transcriptomes, preferably total transcriptomes, of all other members having independently acquired trait A. Preferably the complete sequence of all expressed genes in a selected tissue are compared.

For this, cDNA, preferably total cDNA, is obtained from the at least two members having trait A. Preparation of, cDNA, preferably total cDNA can be done by any suitable method known to the skilled person. Many commercially available kits for cDNA synthesis can be purchased, such as e.g. from ABgene, Ambion, Applied Biosystems, BioChain, Bio-Rad, Clontech, GE Healthcare, GeneChoice, Invitrogen, Novagen, Qiagen, Roche Applied Science, Stratagene, and the like. Such methods are e.g. described in Sambrook et al. (Sambrook, J., Fritsch, E. F., and Maniatis, T., in Molecular Cloning: A Laboratory Manual. Cold Spring Harbor Laboratory Press, NY, Vol. 1, 2, 3 (1989)). The skilled man will also understand that cDNA can be further treated, for example by normalisation to reduce differences in representation of individual cDNA. (Evrogen, etc.).

As indicated, for the method according to the invention it is required that cDNA, preferably total cDNA, of at least two members having trait A, is obtained, sequenced and compared with each other. No comparison with cDNA obtained from (a) member(s) having trait B is, as explained above, required. More preferably, at least 3, 4, 5, 6, 7 or 8 members of the organisms, and each having a trait A are provided/used in the method according to the invention.

As explained above, the current invention is based on the realization that a genetic difference that is associated with a particular character (or trait) can be identified by comparing the sequences of the (preferably whole) transcriptomes of organisms that independently acquired a phenotypical trait (Trait A). Since in a preferred embodiment the members having a trait A are generated by random (chemical) mutagenesis treatment of members having a trait B, the genetic material of such treated organisms will comprise many and random mutations, and of which most will not be associated to the observed traits. Comparing the cDNA, preferably total cDNA, of one member having trait A to the cDNA, preferably total cDNA, of one member having trait B will thus not allow for the identification of the nucleic acid associated with the observed phenotype. It was however realized by the current inventors that when at least two members having trait A are compared with each other, and each preferably having independently acquired said trait A, it becomes possible to identify the nucleic acid that is responsible (or associated) for the character (or trait):

The members having trait A are (independently) derived from the same isogenic source. As a consequence of a mutation treatment of the isogenic source, alterations are randomly introduced in the genetic material. The alterations induced in a first member will be different from the alterations induced in a second member. However, in case both said first member and said second member, as a consequence of the mutagenic treatment, now express a phenotype, i.e. have a trait A, it is extremely likely that in both members the same cDNA will display a single nucleotide polymorphism (SNP). Due to the random process of the mutation treatment, such SNP is very unlikely to be localized at the same position within such cDNA. Said cDNA can now be identified as a cDNA derived from an expressed nucleic acid that is associated with a particular trait or character of the organism under study, as the change that a non-associated cDNA would, in the at least two members having trait A, display such SNPs is near zero.

In other words, the current invention is based on the realization that in the sequences of all cDNA from a “mutant pool”, of e.g. 5 allelic mutations in one gene (trait A), there will be just one individual cDNA that is consistently showing differences in the sequence when compared to each other.

In order to compare the cDNA, preferably total cDNA, of each member having trait A, the cDNA, preferably total cDNA, of the members having trait A needs to be sequenced. Sequencing of cDNA can be done by any suitable method known to the skilled person. However, in particular such methods as described by Margulies M. et al. (http://www.454.com, Genome sequencing in microfabricated high-density picolitre reactors. Nature 437:376-80, 2005] are highly preferably, allowing for rapid and efficient sequencing of the whole transcriptome (all cDNA's). For example, and in a preferred embodiment, the nucleotide sequences of the obtained cDNA fragments are determined by high-throughput sequencing methods, for example like those disclosed in WO 03/004690, WO 03/054142, WO 2004/069849, WO 2004/070005, WO 2004/070007, and WO 2005/003375, by Seo et al. (2004) Proc. Natl. Acad. Sci. USA 101:5488-93, and technologies of Helicos, Solexa, US Genomics, etcetera, which are herein incorporated by reference.

In a next step in the method according to the invention, the obtained sequences of the cDNA, preferably total cDNA, of the members having trait A are compared to the cDNA, preferably total cDNA, of other members having trait A in order to establish single nucleotide polymorphism frequencies in the cDNAs. This determination can be done by any suitable method known to the skilled person, and for example as set-out in the accompanying example.

In short, for example, alignment of the nucleotide sequences of cDNA fragments may be used to collect nucleotide sequences derived from the same transcribed gene (but from the different members having trait A), and to compare these nucleotide sequences. Whether nucleotide sequences are derived from a same transcribed gene can be established based on homology between the sequences. For the purposes of this invention, it is assumed that nucleotide sequences are derived from a same transcribed gene when they are at least 95, 96, 97, 98, 99, 100 percent homologous over a length of at least 30, preferably at least 50, more preferably at least 90, yet more preferably at least 100, 150, 200 nucleotides.

In a next step, and based on the obtained data, cDNA in the collection of cDNA, preferably total cDNA, of the members having trait A having an increased SNP frequency can be identified. In the method according to the invention, individual cDNAs in which at least 2 of the members having trait A contain at least one single nucleotide polymorphism, and preferably wherein these single nucleotide polymorphisms are on different positions in the cDNA are identified. Preferably each of the analyzed members having trait A contains at least one single nucleotide polymorphism in said cDNA, preferably at different positions in the cDNA. For example, in case 4 plants having trait A are analyzed a cDNA might be indentified that contains in the (same; i.e. derived from the same gene in each of the plants) cDNA of the 4 plants a SNP at respectively position 25, 34, 56 and 101 for plants 1, 2, 3, and 4. Alternatively the SNP might be at the same position for some plants, but not for some other plants. For example, the SNP might be a positions 25, 25, 56 and 101, or even 25, 25, 101, and 101, for plants 1, 2, 3, and 4 respectively.

In the method according to the invention, among all the cDNA sequenced in the members having trait A, at least one specific cDNA will carry at least one genetically (preferably non-synonymous) single nucleotide polymorphism (SNP) in at least two, but preferably in each of the members having trait A.

In other words, this specific cDNA will comprise at least one SNP in each of the analyzed members having trait A (not necessarily at the same localization in the corresponding cDNA's). Such cDNA is a likely candidate to be a cDNA derived from a gene involved or responsible for the observed trait A, and this information can subsequently be used to identify the expressed nucleic acid sequence and clone the corresponding gene by methods known to the skilled person.

Optionally and preferably, in a next step of the method according to the invention, individual cDNAs from each of the cDNAs identified in the previous step in which at least two, preferably all, members having trait A have at least one non-synonymous single nucleotide polymorphism are identified.

As will be understood by the skilled person, the at least one SNP found in cDNA identified in the previous step, must very likely be associated with a change in an amino acid in the encoded protein, in order to be involved or responsible or causal for the observed phenotype (trait A). Therefore, identifying the presence of at least one non-synonymous (leading to a codon change, leading to a change in the amino acid that is encoded) further improves the method according to the invention and allows for even more reliable identification of a gene involved in the observed trait A.

Indeed, a skilled person understands the term “non-synonymous” in the context of the current invention as indicating that due to the change of at least one nucleotide in a triplet of bases, a codon change occurs, and wherein the changed codon codifies for a different amino acid in comparison to the original codon.

In case the previous step of identifying cDNAs in which at least two, preferably all, members having trait A, have at least one non-synonymous single nucleotide polymorphism is performed, optionally, but preferably, in a next step, individual cDNA from each of the cDNA in the previous step are indentified in which at least two, but preferably all members having trait A have no more than one non-synonymous single nucleotide polymorphism.

It has been found by the current inventors that indeed such cDNA's are very likely candidates to be derived from a gene responsible for or causal related to the observed phenotype (trait A), thus further improving the method according to the invention.

Further improvements of the method according to the invention can involve identifying individual cDNAs from each of the cDNAs identified in any of the previous steps in which at least two, preferably all members, having trait A have non-synonymous single nucleotide polymorphisms causing amino acid changes at evolutionary conserved amino acid positions, and preferably these new amino acids are chemically distinct from the original non-mutated amino acid (i.e. aromatic versus non-aromatic, polar versus non-polar, negative, positive or neutral at particular pH, positive or negative hydropathy (hydrophilic or hydrophobic) and so on.

Depending on the steps disclosed above performed in the method according to the invention, all individual cDNAs identified in performed steps (d), (f), (g), and/or (h) can be ranked according to the number of steps d-f (as performed) that they comply with. In a next step an expressed nucleic acid sequence which complies with all steps (d), (f), (g), and/or (h), and is thus highest in rank, can be identified as a most likely candidate to be involved, responsible or causally related to the observed phenotype./esp

Based on the above, an expressed nucleic acid sequences can be identified and the gene can be cloned.

In a preferred embodiment, there is provided for a method according to the invention wherein the members of said organism having trait A were obtained by mutagenesis of members of said organism having said trait B, and wherein said members having trait B where isogenic before said mutagenesis treatment.

In an embodiment of the method according to the invention, the at least two members having a trait A of a distinct character can be the consequence of natural variation, i.e. due to natural or spontaneous changes in a nucleic acid in said organism previously manifesting trait B. Such mutations in the genetic information are unintentional, but reveal that the organism carries genetic information responsible for the observed change in phenotype (for example, from trait B to trait A).

Preferably, however, the method according to the invention does not depend on such unintentional and uncontrollable variation in the genetic information, but instead depends on the mutants having a particular trait being the result of deliberate mutagenesis. The skilled person understands how he can mutate the genetic information of any organism, for example by the use of known mutagens. Due to the use of such mutagens, mutations can randomly occur in the genome of members of the organisms. In other words, nucleic acids, like genes, can be tagged (by comparison to non-mutated nucleic acids) with (chemically, biologically or by means of radiation) induced mutations (e.g. point mutations, e.g. by ethylmethanesulfonate).

Examples of “mutagenesis by irradiation” include X-rays, y-rays, UV light, or ionizing particles. Examples of “biological mutagenesis” include, for example, such methods as described in WO0150847, for example using recombinases like recA. Examples of “chemical mutagens” that can be used in the method according to the invention include the application of specific chemicals such as ethylmethanesulfonate (EMS), diethyl sulphate (DES), N-nitroso-N-ethylurea (ENU), diepoxybutane, 2-aminopurine, 5-bromouracil, ethidiumbromide, nitrous acid, nitrosoguanidine, hydroxylamine, sodium azide, or formaldehyde.

Preferably, the method of mutagenesis induces point mutations in the genome, or insertion, substitution or deletion of up to 10 consecutive nucleotides.

The skilled person will understand that within the context of this embodiment the members having trait A and the members having trait B are isogenic (as they are both derived from the same genetic background), except for the mutations introduced by said mutagenesis treatment.

In contrast to classical mapping and tagging strategies with insert mutations however, such small and random mutations are difficult to detect as they do not qualify for any form of specific PCR amplification. However, there are several good reasons to use the above strategy: the mutagenesis can be applied to essentially any species of interest, the spectrum of induced mutants is broader than with tagging approaches, mutagenesis is usually more efficient and second-site mutations are easier to obtain.

Although a wide variety of suitable method of mutagenesis can be employed, preferably, the mutagens are not biological mutagens selected from the group consisting of transposon inserts or T-DNA inserts. Preferably, the used method of mutagenesis introduces point mutations in the genetic material.

In another embodiment there is provided for a method according to the invention and wherein the organism is a plant, preferably a crop plant selected from the group consisting of tomato, pepper, aubergine, lettuce, carrot, onion, leek, chicory, radish, parsley, spinach, melon, and cucumber.

The organism that can be subject to the current invention can be any organism, including bacteria, prokaryotes and eukaryotes. However preferably the organism is an eukaryote, in particular a plant, more in particular a crop plant. Preferably, the plant is a plant belonging to the group consisting of important crop plants, including tomato, pepper, aubergine, lettuce, carrot, but the method can be applied to essentially all others plants.

In particular, the current invention allows for the identification and cloning of nucleic acids, like genes, from crop plants in which mapping and tagging techniques available in the art turn out to be impossible or cumbersome.

In a further embodiment there is provided for a method according to the invention wherein the organism is a plant and wherein prior to step a) a F1 hybrid population that is heterozygous for an allele encoding a recessive trait A and a second allele encoding a dominant trait B is created, and wherein said F1 hybrid population is subjected to mutagenesis and wherein from said F1 population, after said mutagenesis, members having trait A are selected.

Preferably, the allelic series can for example be constructed by a standard genetic approach for selecting new alleles at one locus. This approach consists of creating an F1 population that is heterozygous for one reference allele (which produces the previously recognized recessive phenotype, i.e. trait A, of this locus) and one dominant allele which produces trait B of this locus. Mutagenesis of the F1 will uncover new alleles producing trait A when deleterious mutations occur in allele B. Such mutant F1 plants with trait A will appear at a certain frequency, while the bulk of the F1 population will show trait B. In one example, one can mutagenize seed (or pollen, or any other tissue) of a red-flowered cultivar carrying the dominant allele of the locus (encoding trait B), and use this material for sexual crosses to a non-mutagenized white-flowered cultivar carrying the recessive reference allele of the locus (encoding trait A). In the resulting F1 progeny, the large majority of individuals will be red, except for a few white individuals that carry newly induced, independent mutations in the dominant B allele such that they express trait A. In a second example, one may directly mutagenize F1 material (e.g. seed, or any suitable tissue), and score for the expression of trait A in the resulting individual plants that grow from the mutagenized material. The advantage of examples one and two, is that the use of F1 material heterozygous for the A and B alleles at the locus, allows the newly arising trait A alleles to be assigned with relative certainty to the locus to be cloned. Without the F1 approach, e.g. in F2 populations derived by self-fertilization of the primary mutagenized material, it is theoretically possible that white-flowering mutant plants arise as a result of mutations in different genes than the gene to be cloned. Hence, these newly arising mutations may be non-allelic and will be confounding in the context of the current invention.

The nucleic acid that will be identified by the method according to the invention can subsequently be isolated, cloned (gene), or introduced in a host cell, or used in for example, plant breeding programs.

In summary, the current invention relates to a new strategy for identification, and optional isolation, of a nucleic acid sequence that is expressed in an organism and that is related to a particular phenotype (trait of a character) of said organism. With the method of the current invention it has become possible to, in contrast to known methods in the art, efficiently identify, isolate or clone genes in, for example, organisms like (crop) plants for which no or only limited information with respect to the genome is available. In addition, it is now possible to take advantage of the use of chemicals inducing point mutations as a versatile, broadly applicable method for creating mutagenised populations, for example as much higher mutation frequencies than by using T-DNA or transposon system are generated, thereby reducing the amount of organisms, for example plants, needed in the method. Moreover, the method does not rely on the use or presence of markers in the genome, as it directly detects changes in a gene of interest.

### FIGURES

FIG. 1 is a schematic overview of (an embodiment of) the method according to the invention. In the overview, and applicable to the invention disclosed herein, is shown an isogenic population, depicted by a single individual that is heterozygous for a reference allele carrying a mutation (black circle) at a gene that underlies an observable phenotypic manifestation when homozygous, and which is the gene to be identified by the method according to the invention (FIG. 1A, left image). Following a mutagenic treatment, the population will contain novel alleles (FIG. 1A right image, grey circles) that eliminate or compromise gene function such that the phenotypic effects of the reference allele become observable by loss of heterozygosity. In FIG. 1A, five independent non-synonymous alleles are depicted. FIG. 1B shows the allelic composition of these five plants, selected as a group for the manifestation of the phenotype associated with loss of heterozygosity. In the left image of FIG. 1B, the genotypes of these five plants are depicted as 1-5, with the paternal or maternal allele indicated with the affix “a” or “b”, for a gene that is unrelated to the selected phenotypic manifestation. It can be seen that such gene contains no relevant polymorphisms among plants and parents. In contrast, the selected gene (FIG. 1B right image), which is causally related to the selected phenotype, contains one non-synonymous, or otherwise genetically non-neutral, allele that is shared by all five isogenic plants and is exclusive for one of the parents (in this case, parent “a”). In addition, it contains a novel and different non-synonymous, or otherwise genetically non-neutral, polymorphism in each of the five phenotypically selected plants. This pattern of polymorphism must be exceedingly rare, and can thus be used to identify a nucleic acid sequence (gene) underlying the trait that is defined by the selected phenotype.

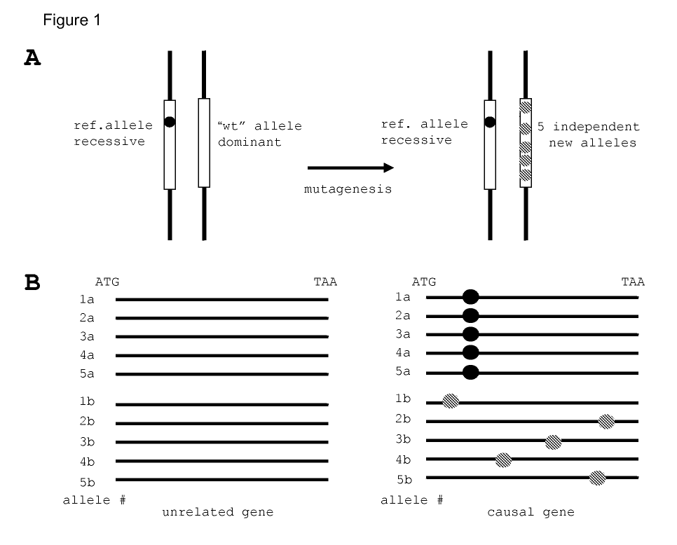

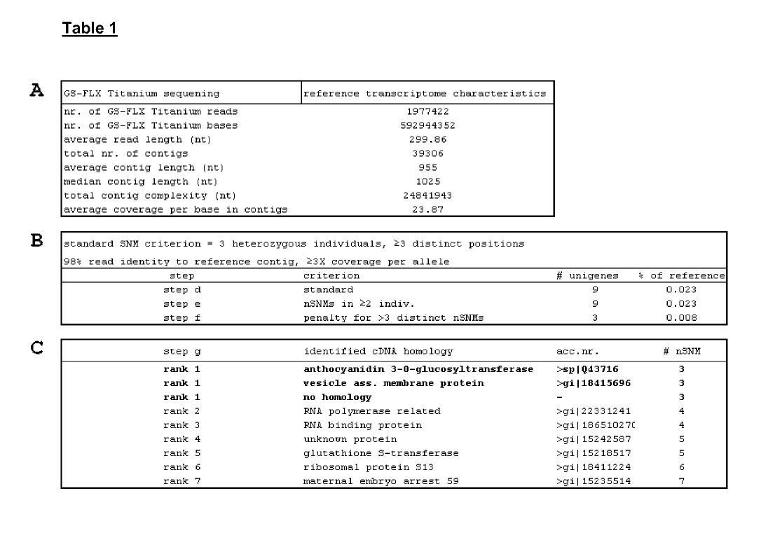

### TABLE 1

A) summary of the combined results (reference transcriptome) after sequencing the transcriptomes of three independent Petunia mutants, having acquired red flower colour by loss-of-heterozygosity.
B) number of unigenes remaining after applying selection criteria on basis of polymorphism patterns in unigenes, according to steps in the present invention. Also given is the enrichment factor after selection, expressed as percentage of the total number of unigenes in the reference transcriptome.
C) ranked list of genes remaining, based on the method according to the invention. Three genes complied perfectly with all criteria applied. The gene known a priori to underlie red flower color is anthocyanidin 3-O-glucosyltransferase shares rank 1 with two other genes.

### EXAMPLE

### De Novo Cloning of a Gene for Flower Pigmentation in Petunia

### Generation of Mutants in a Single Flower Colour Gene.

To demonstrate the described method according to the invention, we aimed at the de novo identification of a previously known flower colour gene (RT) of Petunia hybrida. A magenta flowering F1 hybrid Petunia was produced from a cross between the inbred lines W5 (rt::dTph3; Stuurman and Kuhlemeier 2005, Plant J. 41(6):945-55) and M1 (RT, Snowden KC and Napoli Calif. (1998) Psi: a novel Spm-like transposable element from Petunia hybrida. Plant J. 14:43-54), resulting in a magenta-flowering heterozygous genotype carrying a recessive allele at the gene RT, which codes for UDP rhamnose: anthocyanin-3-glucoside rhamnosyl transferase. In the genetic background used here, null mutants of RT produce red flowers, as a consequence of the accumulation of cyanidin-3-glucoside (Kroon et al. 1994, Plant J. 5(1):69-80; Brugliera et al. 1994, Plant J. 5(1):81-92). Upon mutagenic treatment of F1 hybrid seeds with ethylmethanesulfonate (EMS), some of the resulting plants will carry novel mutations in the wild type allele of RT resulting in loss of heterozygosity and expression of a red corolla colour.

A population of 3600 F1 plants was grown from EMS treated seed, and flower colour was recorded visually on all individual plants. Seven red-flowering mutants were observed. Because red colour is recessive, the expression of this colour in the primary mutagenised population indicates that the red-flowering mutants carried EMS induced point mutations in the wild type copy of the RT gene. These newly isolated mutations are necessarily heterozygous.

To de novo identify the gene underlying the red colour from the newly identified mutant alleles, the complete and normalised transcriptome of corolla limbs at the developmental time point at which anthocyanin pigmentation becomes visible was sequenced in 3 independent mutants. Total RNA of each of the 3 mutants was converted into ds-cDNA using the SMART cDNA synthesis kit (Clontech Company, 1290 Terra Bella Avenue, Mountain View, Calif. 94043, USA). Double stranded (ds-) cDNA was normalised using the Trimmer kit (Evrogen Company, Miklukho-Maklaya str, 16/10, 117997, Moscow, Russia). Normalised cDNA from each individual mutant plant was separately processed for sequencing on a GS-FLX Titanium sequencer (454 Life Sciences). Processing involved fragmentation of cDNA into ˜700 nt, and tagging with 10 bp multiplex identifiers (MID). All processed cDNA samples from all mutants were subsequently pooled, and sequenced with 2 runs of GS-FLX Titanium sequencing. We obtained a total of 1′977′422 reads of average 300 nucleotides length.

All pooled sequence data were trimmed to remove adapter sequences and MID sequences, and subsequently assembled into a non-redundant set of contigs using the assembly program Newbler (Roche 454 Software Release: 2.3-PreRelease-Sep. 14, 2009) using default run assembly settings. An overview of the results is given in table 1A. A total of 39306 unique contigs (average size 955 nucleotides, median size 1025 nucleotides) were obtained, representing the full complement of unigenes expressed in the corolla. Coverage per base was 24×, meaning that the average nucleotide had been sequenced 24 times. This complement is henceforth called “reference”, as it represents the consensus reference transcriptome sequence for subsequent use in the method according to the invention. Similarly, contigs are henceforth called “unigenes”.

The assumption of the method according to the invention is that among the unigenes, one specific unigene will carry a genetically non-synonymous single nucleotide polymorphism (SNP) in most or each of the mutants used to generate transcriptome sequences. In the specific example given here, the gene responsible for generating the red flower colour should be mutated 3 times, once in each individual mutant plant that constituted the cDNA pool. Because random EMS mutation frequencies typically are ˜1 per 200 kilobases, there is a near zero chance that 3 distinct nucleotide mutations will consistently occur in any single unigene, if that unigene is not causally related to the phenotype on basis of which the mutant plants were selected. The particular gene that does conform to this mutation criterion and in which also the base changes are non-synonymous, which means causing an amino acid change in the protein product of that gene, is highly likely the gene causing red flower colour. In the remaining text, base changes are indicated as single nucleotide mutations (SNMs) and non-synonymous single nucleotide mutations as nSNMs. It can be noted that synonymous base changes, that is those that do not cause amino acid changes, can sometimes be genetically non-neutral if they locate e.g. at an intron-exon splice site. Note however, that the method of the invention is robust to such relatively rare cases, as at least a pair or preferentially a group of alleles is used for comparison and because splicing errors will lead to grossly changed cDNAs.

We analyzed the corolla 454 sequence dataset for the occurrence of SNMs in all individual 454 sequence reads by aligning them to the reference corolla transcriptome described above. Alignments of 454 reads were used for SNM analysis only when the reads were at least 98% identical to the reference. Alignments can be done by various bioinformatics methods well known to a person skilled in the art. An SNM is defined in the current example as:

(1) occurring at least 3 times in the complete set of 454 reads (3× coverage)
(2) occurring in only one of the 3 individual mutant plants
(3) occurring in 10%<N<90% of all reads from an individual mutant plant
These three criteria certify that the SNM is not a sequencing error (1), is independent (2), and occurs in heterozygous condition (3). A skilled person will understand that these criteria can be made more stringent or relaxed as prescribed by the number of mutant plants in the pool, and the level of type 1 and type 2 errors in SNM calling. In addition, in this example only G->A or C->T transitions were taken as possible SNMs, as these are the primary mutations induced by the mutagenic agent (EMS) used. A skilled person will also understand that the 98% identity threshold of mapped reads can be made more stringent or relaxed, and that such thresholds can influence the number of SNMs found.

As a next step, we searched for unigenes in which at least three SNMs were present, one for each of the three mutants in the pool, and identified by the SNM calling criteria (1, 2, 3) specified above. Any unigene causally related to the red flowering phenotype must theoretically comply with this rule, provided that sequencing coverage is sufficient to have all SNMs meet the three criteria for SNM calling. Twenty-six unigenes were found to contain at least three SNMs in the coding region, each derived from a different mutant plant. Of these, 20 contained exactly three SNMs whereas 6 contained 4-7 SNMs.

The 26 unigenes that complied with all above described formal genetic patterns of SNMs were subsequently inspected for the occurrence of nSNMs. First, the DNA sequences of the 26 unigenes were converted into amino acid sequences by finding the longest predicted open reading frame (ORF), according to a standard eukaryotic genetic code to translate DNA triplets into amino acids. These ORFs were then compared to the UniProt database of protein sequences, using a standard BLASTp procedure, in order to detect homologous proteins and thereby confirm that the predicted ORF was in the correct biologically meaningful translational reading frame. The protein sequences of each of the 26 reference unigenes were then compared to their counterpart protein sequences of the mutant plants containing SNMs. If an amino acid change occurred in the codon that comprises an SNM, that SNM was marked as nSNM. By definition, only nSNMs can cause phenotypic change within coding regions. Therefore, any of the 26 unigenes that did not contain at least three nSNMs and with each mutant plant containing at least one nSNM, was discarded as a possible candidate causing the red flowering phenotype. Following this operation, 9 unigenes remained on the list of candidates (table 1B).

The remaining set of 9 unigenes was subsequently subjected to a ranking procedure on formal genetic criteria. A penalty was given for the presence of more than three nSNMs, where the perfect pattern is exactly three as dictated by the fact that exactly three mutant plants were used in the analysis. The more nSNMs, the lower the rank. Following this operation, 3 unigenes shared rank 1. The full list of 9 unigenes and their ranking is given in table 10, including the best BLAST match to the UniProt database. It can be seen that the RT gene, which was a priori known to cause the red flowering phenotype and codes for UDP rhamnose: anthocyanin-3-glucoside rhamnosyl transferase, has rank 1.

From these data it is concluded that the method according to the invention allows the identification of a single gene that causes a mutant phenotype among a small set of candidates within a background of 39306 unigenes. A person skilled in the art will understand that the method according to the invention can be performed in any species other than Petunia. A skilled person will also understand that the method can be performed with different numbers of mutant individuals per pool, different identity percentages during the mapping of reads to a reference transcriptome, different stringencies of coverage per SNM, or other scalable settings applied to the search. It is also clear that additional objective criteria for ranking candidates can be used, either singly or combined, which may include:

the base calling quality scores of possible SNMs obtained from the raw sequencing data

the degree of match to a perfect distribution of SNMs over the individual alleles in the pool according formal genetic predictions that follow from the composition of the pools and the heterozygoyus or homozygous state of the used mutant plants.

the chemical similarity score of amino acid replacements due to nSNMs compared to the wild type amino acid

### Materials and Methods

1) Preparation of cDNA for GS-FLX Titanium Sequencing

To prepare cDNA samples suitable for analysis, the following steps were followed.

I. Total RNA isolation of each mutant plant's petal tissue at the onset of pigmentation, using the Qiagen RNeasy Plant Mini kit (Qiagen Company, 27220 Turnberry Lane Valencia, Calif. 91355) according to the manufacturer's instructions.
II. SMART double stranded (ds-) cDNA synthesis according to the SMART cDNA synthesis for Library Construction protocol (Clontech Company, 1290 Terra Bella Avenue, Mountain View, Calif. 94043, USA) according to the manufacturer's instructions.
III. cDNA normalisation using the TRIMMER cDNA Normalization kit (Evrogen Company, Miklukho-Maklaya str, 16/10, 117997, Moscow, Russia) following the manufacturer's instructions.
IV. Removal of polyA tail from ds-cDNA by BAL31 exonuclease (New England Biolabs, 240 County Road, Ipswich, Mass. 01938-2723, USA) using the following reaction set up:

| | | |
|---|---|---|
| 2 x BAL31 reaction buffer | 25.0 | μl |
| 1 U/μl BAL-31 | 0.25 | μl |
| cDNA (1 μg) | x | μl |
| double distilled water | y | μl |
| Total | 50 | μl |

BAL31 exonucleolytic reactions were allowed to proceed according the manufacturer's instructions, until ˜50 nucleotides were removed from the 5′ and 3′ ends of cDNA, as judged by agarose gel electrophoresis and observing average molecular size shifts of 50+50=100 basepairs. The treated cDNA samples were then purified using a Qiagen PCR purification kit according the manufacturer's instruction (Qiagen Company, 27220 Turnberry Lane Valencia, Calif. 91355).
V. BAL31 treated cDNA samples were divided into 15 sub-samples, and each subsample was fragmented by digestion with one restriction enzyme from the following list, with their DNA recognition sequences in brackets: Accl (GTMKAC), Alul (AGCT), Apol (RAATTY), Aval (CYCGRG), Avall (GGWCC), BsaAl (YACGTR), BsiEl (CGRYCG), BstYl (RGATCY), Haell (RGCGCY), Hpy99I (CGWCG), Msel (TTAA), Nspl (RCATGY), Rsal (GTAC), Sfcl (CTRYAG), Styl (CGWWGG). These restriction enzymes were chosen for their ability to generate fragments is a size range suitable for GS-FLX Titanium sequencing, while securing a good coverage of cDNA and a good probability of overlap among fragments to allow contig building. The reactions were allowed to proceed to completion and subsequently inactivated, according the enzyme manufacturer's instructions. All enzymes were obtained from New England Biolabs (240 County Road, Ipswich, Mass. 01938-2723, USA). Following complete digestion, all 15 restriction reactions were pooled per single mutant plant, and purified with the Qiagen PCR purification kit according the manufacturer's instruction (Qiagen Company, 27220 Turnberry Lane Valencia, Calif. 91355).
VI 3-5 μg of purified cDNA, from each mutant plant individually, was used to generate a GS-FLX Titanium library for sequencing, using the GS-FLX Titanium General Library Preparation Kit (Roche Diagnostics Corporation, Roche Applied Science, P.O. Box 50414, 9115 Hague Road, Indianapolis, Ind. 46250-0414, USA) according to the manufacturer's instructions. The individual libraries were prepared with distinct multiplex identifiers (MID). The MID distinguishable libraries of the three mutant plant used in this work were then pooled and sequenced using two runs of GS-FLX Titanium.

### Bioinformatic Analysis

### Pre-Processing of Raw GS-FLX Titanium Reads

Reads were pre-processed to remove MID and adapter sequences provided in the GS-FLX Titanium General Library Preparation Kit (Roche Diagnostics Corporation, Roche Applied Science, P.O. Box 50414, 9115 Hague Road, Indianapolis, Ind. 46250-0414, USA).

### Assembly of a Reference Transcriptome Sequence

Assembly was done with the Newbler assembly software of 454 Life Sciences (454 Life Sciences, 15 Commercial Street, Branford, Conn. 06405, USA) with the pre-processed sequences as input, using 454's default run assembly settings.

### Analysis

Pre-processed reads were mapped on the assembled reference contigs, using a minimum identity of 98%. Candidate unigenes were selected by having at least three SNMs on different positions in the unigene and by association with a distinct mutant plant individual. SNMs were called from a minimum coverage of 3×. The nature of the SNM was forced to be G->A or C->T. Only one, and heterozygous, mutant plant was allowed per SNM position. Nucleotide positions were considered homozygous, if the same nucleotide was seen in ≧90% of cases per individual plant.

1. A method for identification, and optional isolation, of an expressed nucleic acid sequence that is associated with a character of an organism, characterized in that the method comprises the following steps:
a) Providing at least two members of said organism having a trait A of said character, and wherein said members having trait A are derived, preferably independently derived, from isogenic member(s) of said organism having trait B, and wherein trait A and trait B are different;
b) Obtaining cDNA from each of the members of step a) having trait A and determining nucleotide sequences of at least part of, preferably of each of the individual cDNA's obtained from the members having trait A;
c) Determining single nucleotide polymorphism positions in each individual cDNA of members having trait A by comparison to corresponding cDNA of other members having trait A;
d) Identifying individual cDNAs in which at least 2 of the members having trait A, preferably all of the members having trait A, contain at least one single nucleotide polymorphism, and preferably where these single nucleotide polymorphisms are on a different position in the cDNA for said at least two, preferably all members having trait A;
e) Optionally, verifying for each of the cDNAs indentified in step d) that it is associated with a character of an organism.

2. Method according to claim 1 wherein the members of said organism having trait A were obtained by mutagenesis of members of said organism having said trait B, and wherein said members having trait B where isogenic before said mutagenesis treatment.

3. Method according to claim 2 wherein the method of mutagenesis induces point mutations.

4. Method according to claim 2 wherein mutagenesis is performed by use of a non-biological mutagens.

5. Method according to claim 1 wherein the organism is a plant, preferably a crops plant selected from the group consisting of tomato, pepper, aubergine, lettuce, carrot, onion, leek, chicory, radish, parsley, spinach, melon, cucumber.

6. Method according to claim 1 wherein the organism is a plant and wherein prior to step (a) a F1 hybrid population that is heterozygous for an allele encoding a recessive trait A and a second allele encoding a dominant trait B is created, and wherein said F1 hybrid population is mutagenized and wherein from said F1 population, after said mutagenesis, members having trait A are selected.
